(19) United States
(12) Patent Application Publication
(10) Pub. No.: US 2024/0416078 A1
(43) Pub. Date: Dec. 19, 2024

(54) Instrument Advancement Device with Enhanced Instrument Viewing

(71) Applicant: Becton, Dickinson and Company, Franklin Lakes, NJ (US)

(72) Inventors: Curtis H. Blanchard, Herriman, UT (US); Weston F. Harding, Lehi, UT (US); Yiping Ma, Layton, UT (US); Marshall Scott Butler, Provo, UT (US); Adam J. Boud, Bluffdale, UT (US); Austin Paul Newton, Layton, UT (US)

(21) Appl. No.: 18/743,360

(22) Filed: Jun. 14, 2024

Related U.S. Application Data
(60) Provisional application No. 63/508,597, filed on Jun. 16, 2023.

(51) Int. Cl. A61M 25/01; A61B 5/15; A61M 25/00

(52) U.S. Cl. CPC A61M 25/0105; A61B 5/150824; A61B 5/150992; A61M 25/0045; A61M 2025/0008

(57) ABSTRACT

Provided herein is an instrument delivery device for use with an intravenous catheter assembly. The instrument delivery device includes a housing having a proximal end portion and a distal end portion, with the housing including a first housing portion and a second housing portion arranged side-by-side and coupled together to define an inner volume. The instrument delivery device also includes an instrument movably received within the inner volume, a coupling device positioned at the distal end portion of the housing and configured to couple the housing to an access connector of an intravenous catheter assembly, and an advancement member configured to move relative to the housing to advance a distal end of the instrument beyond the distal end portion of the housing and into the intravenous catheter assembly. The housing comprises a visualization aid configured to improve visibility of the instrument within the inner volume of the housing.

### CROSS-REFERENCE TO RELATED APPLICATION

The present application claims priority to U.S. Provisional Application Ser. No. 63/508,597 entitled “Instrument Advancement Device with Enhanced Instrument Viewing” filed Jun. 16, 2023, the entire disclosure of which is hereby incorporated by reference.

### BACKGROUND OF THE INVENTION

### Field of the Invention

Provided herein are instrument advancement devices for use in vascular access, and, in particular, instrument advancement devices incorporating components or features for enhanced instrument viewing.

### Description of Related Art

Catheters are frequently utilized to administer fluids into and out of the body. Patients in a variety of settings, including in hospitals and in home care, receive fluids, pharmaceuticals, and blood products via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples.

As known in the art, instrument advancement devices are often used in connection with an in-dwelling PIVC to facilitate blood draw administer fluids, or for sensing or other purposes. When such instrument advancement devices are used to facilitate blood draw, the devices (i.e., “blood draw devices) have focused on the ability to reliably collect a high-quality blood sample and reduce hemolysis. The main method by which these instrument advancement devices work is by employing an introducer for inserting a catheter, probe, tube, or other instrument through the catheter lumen of the PIVC, with the introducer being attached to a catheter adapter of the PIVC that provide for insertion of the catheter. For example, the catheter adapter may include a needleless access connector thereon by which the catheter may be introduced to provide access to the PIVC and into the patient's vasculature. If performing a blood draw, a syringe or vacutainer may then be used to collect blood samples without needing to subject the patient to additional needle sticks.

The introducer of an instrument advancement device typically includes a housing, an instrument (i.e., a catheter or other probe, tube, or instrument) movable within the housing so as to be extendable out therefrom for advancement into the in-dwelling PIVC, and an advancement member that may be actuated by an operator relative to the housing. That is, the advancement member may be moved distally by the operator to cause a corresponding movement of the instrument relative to the housing, such that the instrument may be advanced out from the housing and into the in-dwelling PIVC.

It is recognized that instrument advancement devices may be employed in many different environments and in varying conditions. For example, for various reasons it may be required or desired to perform a blood draw at night or in surroundings where there is a lack of light (e.g., for the well-being of the patient, such as to enable them to sleep) thereby resulting in reduced/poor visibility for a clinician operating the introducer. Under such conditions, it may be difficult for the clinician to visualize the catheter (or other instrument) as it is advanced through the housing, such that the clinician may not be able to determine the position of the distal tip of the catheter and its position relative to the instrument advancement device and in-dwelling PIVC. In some instances, it is recognized that the instrument (i.e., catheter or probe) may bow during its advancement such as when encountering an obstruction or other resistance, and it is important to be able to observe such potential bowing during advancement to prevent kinking of the probe that might result in damage thereto and/or lead to difficulties in performing the blood draw.

Accordingly, a need exists in the art for an instrument advancement device and introducer thereof that provides for improved visibility during operation of the device, including being able to visualize the distal end of a catheter or other instrument as it is advanced by operation of the introducer.

### SUMMARY OF THE INVENTION

Provided herein is an instrument delivery device for use with an intravenous catheter assembly. The instrument delivery device includes a housing having a proximal end portion and a distal end portion, with the housing including a first housing portion and a second housing portion arranged side-by-side and coupled together to define an inner volume. The instrument delivery device also includes an instrument movably received within the inner volume, a coupling device positioned at the distal end portion of the housing and configured to couple the housing to an access connector of an intravenous catheter assembly, and an advancement member configured to move relative to the housing to advance a distal end of the instrument beyond the distal end portion of the housing and into the intravenous catheter assembly. The housing comprises a visualization aid configured to improve visibility of the instrument within the inner volume of the housing.

In some embodiments, each of the first housing portion and the second housing portion is formed of a transparent or semi-transparent material, with each of the first housing portion and the second housing portion comprises a top region, a bottom region, and a side region.

In some embodiments, the visualization aid comprises a coating applied to the first housing portion, the coating applied to an interior surface of the first housing portion on at least one side region.

In some embodiments, the coating comprises an opaque coating having a first color, and wherein the instrument has a second color that is darker than the first color, such that the coating is contrasted from the instrument.

In some embodiments, the coating forms a one-way mirror that may be looked through from a first side, but has an opaque or reflective nature on an opposing second side.

In some embodiments, the visualization aid comprises the side region of each of the first housing portion and the second housing portion.

In some embodiments, the side region of each of the first and second housing portions has a thickness that is less than a thickness of the top region and the bottom region, to facilitate better visualization of the instrument through the side region.

In some embodiments, the side region of each of the first and second housing portions has an interior surface and an exterior surface, with each of the interior surface and an exterior surface comprising a flat surface.

In some embodiments, the side region of each of the first and second housing portions has an interior surface and an exterior surface, with the exterior surface comprising a curved surface and the interior surface comprising a flat surface or a convex surface having a larger radius than the curved exterior surface.

In some embodiments, the side region of each of the first and second housing portions comprises a smoothed exterior surface free of any steps or indents, the smooth exterior surface minimizing refraction and reflecting of light as it passes through the side region.

In some embodiments, the visualization aid comprises a sticker applied to the side region of the first housing portion, the sticker applied to an exterior surface of the side region and having a first color, and wherein the instrument has a second color that is darker than the first color, such that the instrument is contrasted from the sticker.

In some embodiments, the visualization aid comprises a sleeve secured to an exterior surface of the first housing portion, the sleeve having a first color, and wherein the instrument has a second color that is darker than the first color, such that the instrument is contrasted from the sleeve.

In some embodiments, the visualization aid comprises an insert positioned adjacent an interior surface of the housing, within the inner volume, the insert having a first color, and wherein the instrument has a second color that is darker than the first color, such that the instrument is contrasted from the insert, and wherein the insert is movable from a first position adjacent the side region of the first housing portion to a second position adjacent the side region of the second housing portion via a twist knob located on the housing.

In some embodiments, the visualization aid comprises an opaque material from which the first housing portion is formed, with the second housing portion formed of a transparent or semi-transparent material, the opaque material having a first color, and wherein the instrument has a second color that is darker than the first color, such that the first housing portion is contrasted from the instrument.

In some embodiments, the visualization aid comprises a semi-transparent material from which the first housing portion and the second housing portion are formed, the semi-transparent material having a tinted first color, and wherein the instrument has a second color that is darker than the first color, such that the first housing portion and the second housing are contrasted from the instrument.

In some embodiments, at least one of the first housing portion and the second housing portion comprises a co-molded component having a top region, a bottom region, and a side region, wherein the top region and the bottom region are formed of an opaque material and the side region is formed of a transparent material, and wherein the visualization aid comprises the side region.

In some embodiments, each of the first housing portion and the second housing portion is formed of an opaque material, with each of the first housing portion and the second housing portion comprising a top region, a bottom region, and a side region, and wherein the visualization aid comprises a window formed in the side region of at least one of the first housing portion and the second housing portion, with a transparent member secured within the window.

Also provided herein is an instrument delivery device for use with an intravenous catheter assembly. The instrument delivery device includes a housing having a proximal end portion and a distal end portion, with the housing including a first housing portion and a second housing portion arranged side-by-side and coupled together to define an inner volume. The instrument delivery device also includes an instrument movably received within the inner volume, a coupling device positioned at the distal end portion of the housing and configured to couple the housing to an access connector of an intravenous catheter assembly, and an advancement member configured to move relative to the housing to advance a distal end of the instrument beyond the distal end portion of the housing and into the intravenous catheter assembly. The instrument comprises a visualization aid configured to improve visibility of the instrument within the inner volume of the housing.

In some embodiments, the visualization aid comprises a plurality of stripes formed on the instrument, each of the plurality of stripes extending circumferentially about the instrument.

In some embodiments, the visualization aid comprises a coloring of the instrument, wherein the coloring of instrument is darker than a coloring of the housing, such that the instrument is contrasted from the housing.

In some embodiments, the visualization aid comprises a positioning of the instrument within the inner volume of the housing, the instrument positioned so as to be off-center relative to a center line of the inner volume extending longitudinally between the distal end and the proximal end of the housing.

### BRIEF DESCRIPTION OF THE DRAWINGS

FIG. 1 is a perspective view of a catheter system, in accordance with an aspect of the disclosure;

FIG. 2 is a perspective view of a blood draw device useable with the catheter system of FIG. 1, in accordance with an aspect of the disclosure;

FIG. 3 is an exploded view of the blood draw device of FIG. 2, in accordance with an aspect of the disclosure;

FIG. 4 is a side view of the blood draw device of FIG. 2, showing the catheter tube in a first, retracted position;

FIG. 5 is a side view of the blood draw device of FIG. 2, showing the catheter tube in a second, extended position;

FIGS. 6A and 6B are perspective views of portions of the blood draw device housing of the blood draw device of FIG. 2, including a visualization aiding element provided thereon, in accordance with an aspect of the disclosure;

FIG. 7 is a cross-section perspective view of the blood draw device of FIG. 2, including a visualization aiding element provided thereon, in accordance with an aspect of the disclosure;

FIGS. 8A and 8B are perspective views of portions of the blood draw device housing of the blood draw device of FIG. 2, including a visualization aiding element provided thereon, in accordance with an aspect of the disclosure;

FIG. 9 is a cross-section perspective view of the blood draw device of FIG. 2, including a visualization aiding element provided thereon, in accordance with an aspect of the disclosure;

FIG. 10 is a cross-section perspective view of the blood draw device of FIG. 2, including a visualization aiding element provided thereon, in accordance with an aspect of the disclosure;

FIG. 11 is a cross-section perspective view of the blood draw device of FIG. 2, including a visualization aiding element provided thereon, in accordance with an aspect of the disclosure;

FIG. 12 is a cross-section perspective view of the blood draw device of FIG. 2, including a visualization aiding element provided thereon, in accordance with an aspect of the disclosure;

FIG. 13 is a cross-section perspective view of the blood draw device of FIG. 2, including a visualization aiding element provided thereon, in accordance with an aspect of the disclosure;

FIG. 14 is a cross-section perspective view of the blood draw device of FIG. 2, including a visualization aiding element provided thereon, in accordance with an aspect of the disclosure;

FIG. 15 is a cross-section perspective view of the blood draw device of FIG. 2, including a visualization aiding element provided thereon, in accordance with an aspect of the disclosure;

FIG. 16 is a cross-section perspective view of the blood draw device of FIG. 2, including a visualization aiding element provided thereon, in accordance with an aspect of the disclosure;

FIG. 17 is a side perspective view of a portion of the blood draw device of FIG. 2, including a visualization aiding element provided thereon, in accordance with an aspect of the disclosure;

FIG. 18 is a perspective view of the blood draw device of FIG. 2, including a visualization aiding element provided thereon, in accordance with an aspect of the disclosure;

FIG. 19 is a perspective view of a first housing portion of the blood draw device of FIG. 2, including a visualization aiding element provided thereon, in accordance with an aspect of the disclosure;

FIG. 20A is a perspective view of the blood draw device of FIG. 2, including a visualization aiding element provided thereon, in accordance with an aspect of the disclosure;

FIG. 20B is a cross-section perspective view of the blood draw device of FIG. 20A;

FIG. 21 is an exploded view of the blood draw device of FIG. 2, including a visualization aiding element provided thereon, in accordance with an aspect of the disclosure;

FIG. 22 is a cross-section perspective view of the blood draw device of FIG. 2, including a visualization aiding element provided thereon, in accordance with an aspect of the disclosure; and

FIG. 23 is a perspective view of a catheter system, in accordance with another aspect of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device being manipulated by the user would be the proximal end of the device.

Provided herein are devices and systems for introducing instruments through indwelling catheters, such as peripheral intravenous catheters (PIVCs), peripherally inserted central catheters (PICCs), central venous catheters (CVCs), and midline catheters. While certain catheter assemblies are shown in the accompanying figures and described below, those of skill will appreciate that a disinfecting probe as described herein may be useful in any number of different catheter assembly configurations.

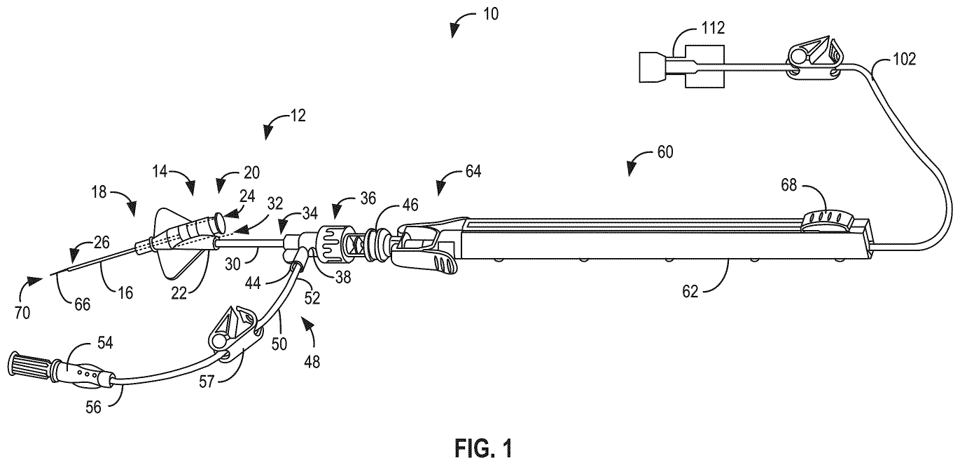

Referring now to FIG. 1, shown is a non-limiting embodiment of a catheter system 10 for facilitating delivery of an instrument into an in-dwelling catheter, such as may be desirable for blood collection from the patient. Catheter system 10 may include a catheter assembly 12 having a catheter adapter 14 and associated catheter 16. The catheter adapter 14 may include a distal end 18 and a proximal end 20. In some embodiments, the catheter adapter 14 may include an additional adapter port 22 that may disposed between the distal end 18 and the proximal end 20 or disposed at the proximal end 20. The catheter adapter 14 may include a first lumen 24 extending through the distal end 18 and the proximal end 20, and the first lumen 24 may be sealed at proximal end 20 of catheter adapter 14. The catheter 16 may be formed of any suitable material and may be of any useful length, as known to those of skill in the art. Catheter 16 extends from the distal end 18 of catheter adapter 14 and may be placed into the vasculature of the patient, with a distal end or tip 26 of the catheter 16 positioned appropriately within a vein to enable a blood draw from the patient.

In some non-limiting embodiments or aspects, the catheter assembly 12 may include a first fluid conduit 30 extending from the port 22. First fluid conduit 30 may be formed of any suitable material known to those of skill in the art and may have a distal end 32 and a proximal end 34. The distal end 32 of first fluid conduit 30 is coupled to port 22, while the proximal end 34 of first fluid conduit 30 may be coupled to a connector 36. Connector 36 may be a t-connector (e.g., one side port arranged at a 90 degree angle relative to a longitudinal axis of connector 36), a y-connector (e.g., one side port arranged at a 25, a 60, or a 75 degree angle relative to a longitudinal axis of connector 36), or any other type of connector known in the art. The connector 36 includes a second lumen 38 therethrough, having any number of branches suitable for the type of connector, such as a branch extending between distal and proximal ends of connector 36 and a branch provided to a port 44 of the connector 36.

In some non-limiting embodiments or aspects, catheter assembly 12 may include a needleless access connector 46 coupled to the proximal end 42 of connector 36, with the needleless access connector 46 providing an access port to the catheter assembly 12. The needleless access connector 46 may be configured as a split-septum connector or self-healing septum connector, as examples. In the illustrated embodiment, the access port provided by needleless access connector 46 is a near-patient access port close to the insertion site of the catheter 16, but it is recognized that an access port could be provided at other alternative locations close enough to the insertion site that allow for advancement of a blood draw catheter tube into the indwelling catheter 16 and out beyond the distal tip thereof. For example, an access port that provides for advancement of a blood draw catheter tube into the indwelling catheter 16 could be located on another connector, such as a proximal connector on an extension set (as described below) of the catheter assembly 12.

In some non-limiting embodiments or aspects, catheter assembly 12 may also include an extension set 48 coupled to the port 44 of the connector 36. The extension set 48 may include a second fluid conduit 50 coupled to port 44 at end 52 of the conduit 50 and a luer connection 54 at opposing end 56, with a clamp 57 provided on second fluid conduit 50 that allows for occlusion thereof.

Catheter system 10 further includes an instrument delivery device 60 that may be operated to introduce an instrument into the catheter assembly 12 and into the vasculature of the patient. In some embodiments, and as referred to hereafter, the instrument delivery device 60 may comprise a blood draw device (“blood draw device 60”) that introduces a catheter tube through the catheter assembly 12 and into the patient vasculature to obtain a blood sample from the patient. However, it is recognized that the instrument delivery device 60 could instead introduce a guidewire, probe, or other sensor into the patient vasculature, according to aspects of the disclosure, and thus it is to be understood that the below description of a blood draw device should not limit the scope of the disclosure.

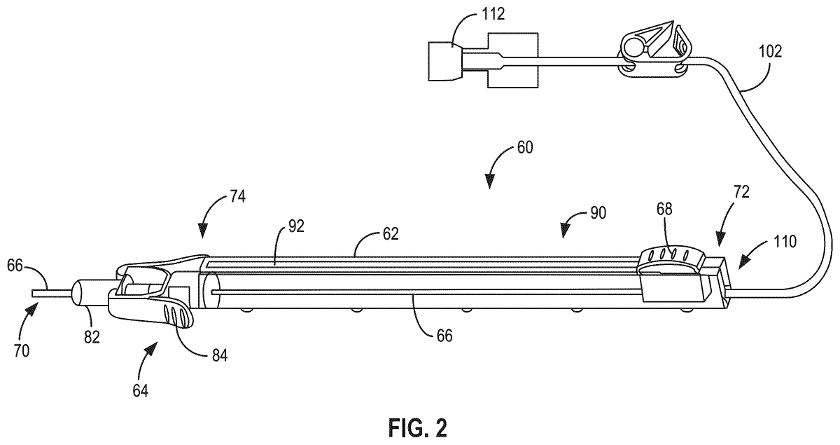

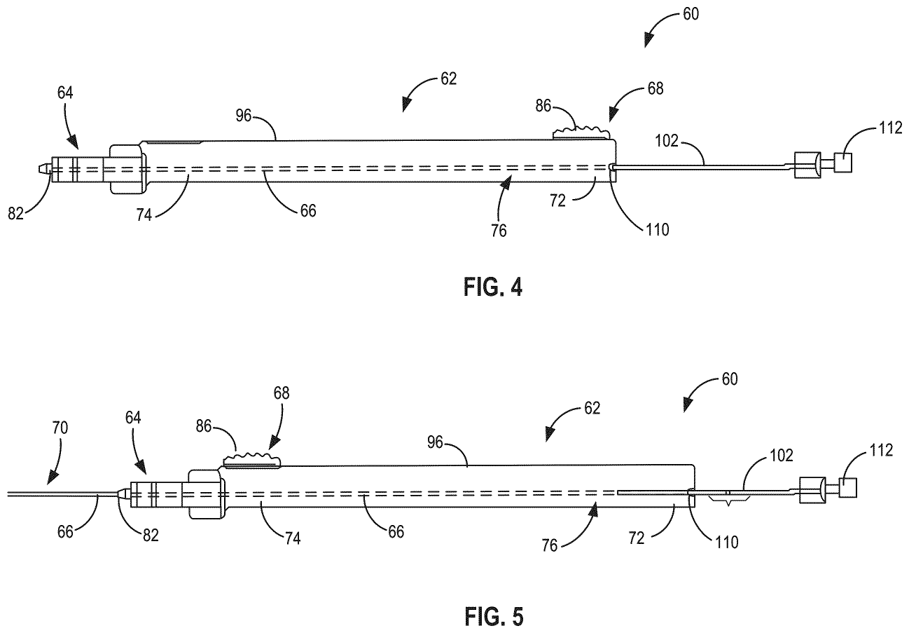

As shown in FIG. 1 and in further detail in FIGS. 2-4, according to a non-limiting embodiment, the blood draw device 60 includes at least a housing 62, a coupling device 64, a catheter tube 66, and an advancement member 68. As will be described in further detail below, the catheter tube 66 is moveable within the housing 62 so as to provide for advancement of a portion of the catheter tube 66 from a first or retracted position inside the housing 62 (FIG. 4 to a second or advanced position outside of the housing 62 (FIG. 5), such that a distal end thereof may be routed into the catheter assembly 12. Once a portion of the catheter tube 66 has been routed into the catheter assembly 12 and out past the distal tip 26 of indwelling catheter 16, the catheter tube 66 may enable collection of a blood sample.

According to embodiments, the catheter tube 66 is sized to enable introduction thereof into the fluid path (i.e., into a lumen of catheter 16, lumen 24 of catheter adapter 14, and first fluid conduit 30) of catheter assembly 12 and for advancement therethrough. Accordingly, the catheter tube 66 can have an outer diameter (e.g., between a 10-gauge and a 30-gauge) that is smaller than the smallest lumen of the catheter assembly fluid path. The catheter tube 66 can have a length that is sufficient to place a distal end 70 of the catheter tube 66 in a desired position within the fluid path of the catheter system 10. Thus, in one embodiment, the catheter tube 66 may have a length sufficient to provide for advancement of the distal end 70 thereof out from the housing 62 and through the catheter assembly (i.e., through connector 36, fluid conduit 30, catheter adapter 14 and catheter 16), and all the way out past the distal tip 26 of catheter 16.

As shown in FIGS. 1-5, the housing 62 of blood draw device 60 can be an elongate member having a proximal end 72 and a distal end 74 and defining an inner volume 76. In some embodiments, the housing 62 may be formed of a pair of housing portions 78a, 78b that are arranged side-by-side and coupled together to define the inner volume 76. The housing 62 may include one or more features or surface finishes on an outer surface thereof that can be arranged to increase the ergonomics of the blood draw device 60, which in some instances can allow a user to manipulate the blood draw device 60 with one hand (i.e., single-handed use). Additionally, the housing 62 may be constructed of a transparent or translucent material that provides for at least partial visibility into the inner volume 76 thereof, such that positioning of catheter tube 66 within the housing 62 can be ascertained by a clinician, as explained in further detail below.

The coupling device 64 of blood draw device 60 is provided at the distal end 74 of the housing 62, with the coupling device 64 providing for reversible coupling of the blood draw device 60 to catheter assembly 12, such as via needleless access connector 46 as shown in FIG. 1. In some embodiments, the coupling device 64 is configured as a lock 80 that includes a blunted cannula 82 and locking arms 84 for coupling to the needleless access connector 46 of catheter assembly 12, with the blunted cannula 82 and locking arms 84 forming three points of contact therewith. However, those of skill will appreciate that any connection or coupling, for example a luer, can be used, so long as the distal end 70 of catheter tube 66 may pass through the coupling device 64 to catheter assembly 12.

The advancement member 68 of blood draw device 60 includes a first portion 86 and a second portion 88. The first portion 86 is movably disposed along an upper surface 90 of the housing 62 and the second portion 88 is movably disposed within the inner volume 76 of the housing 62. The arrangement of the advancement member 68 and the housing 62 is such that a connecting portion (not shown) of the advancement member 68 that joins the first and second portions 86, 88 is seated within a slot 92 formed in the upper surface 90 of the housing 62—the slot 92 generally extending between the proximal and distal ends 72, 74 of the housing 62. As the first and second portions 86, 88 are joined together, movement of the first portion 86 along the upper surface 90 of the housing 62 results in a corresponding movement of the second portion 88 within the inner volume 76.

As shown in FIGS. 1-5, the first portion 86 of the advancement member 68 may be configured as a tab having a contact surface 94a engageable by a user and an underside 94b that is in contact with the outer surface 90 of the housing 62. In such embodiments, the upper surface 90 of the housing 62 can include a track 96, for example, a set of ribs, ridges, bumps, grooves, and/or the like along which the underside 94b of tab or protrusion advances when the advancement member 68 is engaged by a user. In this manner, a user can engage the first portion 86 of the advancement member 68 and can move the advancement member 68 relative to the housing 62.

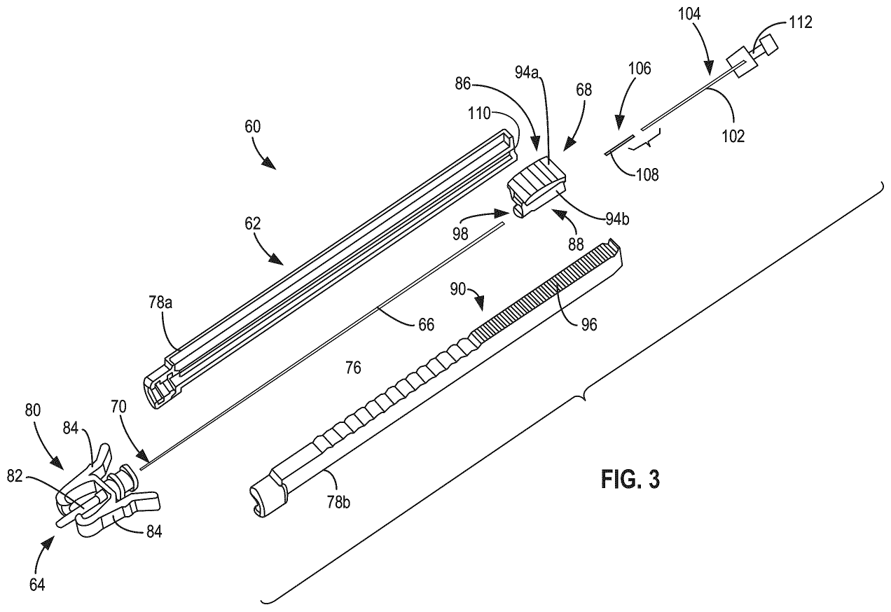

As shown in FIG. 3, the second portion 88 includes an opening 98 extending therethrough that is configured to grip or retain a portion of the catheter tube 66. Due to a portion of the catheter tube 66 being retained within the opening 98 of second portion, 94, movement of the advancement member 68 relative to housing 62 causes a corresponding movement of the catheter tube 66 relative to the housing 62. In this manner, the distal end 70 of the catheter tube 66 can be selectively moved out of or back into the inner volume 76 of the housing 62 as desired, such as advancing the distal end 70 of the catheter tube 66 out of the housing 62 when the blood draw device 60 has been coupled to the catheter assembly 12 and collection of a blood sample is to be performed.

As further shown in FIGS. 1-5, blood collection device 60 includes a secondary catheter 102 provided at the proximal end 72 of the housing 62. The secondary catheter 102 has a proximal end portion 104 and a distal end portion 106 and defines a lumen 108. A portion of the secondary catheter 102 is disposed within and extends through an opening 110 formed in the proximal end 72 of housing 62. As such, the proximal end portion 104 is at least partially disposed outside of the housing 62 and the distal end portion 106 is at least partially disposed within the housing 62, with the distal end portion 106 coupled to the second portion 88 of the advancement member 68. In some embodiments, the secondary catheter 102 can have a larger diameter than the catheter tube 66, which can function to limit, reduce, and/or substantially prevent hemolysis of a volume of blood as the volume of blood flows through the catheter 66 and the secondary catheter 102. According to embodiments of the disclosure, the proximal end portion 104 of the secondary catheter 102 is coupled to and/or otherwise includes a coupler 112 configured to mate with a collection device (not shown) that is useable with the blood draw device 60 to collect a blood sample for subsequent analysis. According to some embodiments, the coupler 112 may be configured as a luer connection (i.e., a female luer connection) configured to mate with a corresponding luer connection (i.e., a male luer connection) of the collection device.

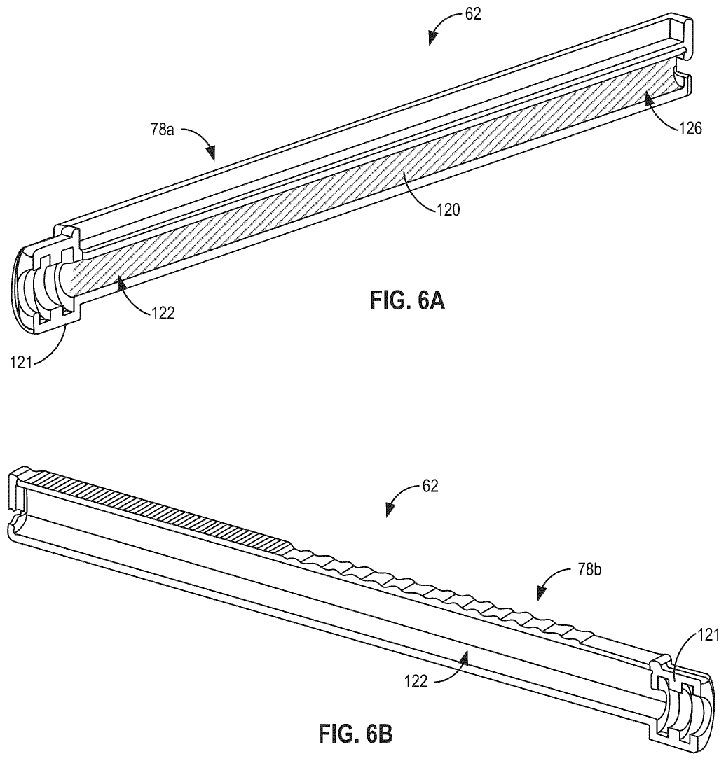

According to aspects of the disclosure, and referring now to FIGS. 6-22, blood draw device 60 is configured to incorporate components or features therein that provide improved viewing of the catheter tube 66 during operation of the device 60—i.e., a “visualization aid.” That is, one or more components or features of the device 60 may be configured to improve visualization of the catheter tube 66 during operation of the blood draw device 60, such that positioning of the catheter tube 66 and/or any bowing or other deformation of the catheter tube 66 can be identified as the catheter tube 66 is advanced and/or retracted relative to the housing 62 and into catheter assembly 12. As described in further detail below, in accordance with some aspects of the disclosure, at least a portion of the housing 62 of blood collection device 60 may be specifically modified to provide for improved viewing of the catheter tube 66, including modifications to the structure of the housing 62 and/or the material from which housing 62 is formed, the application of coating(s) to the housing 62, or the addition of components onto the housing 62. In accordance with further aspects of the disclosure, components within the housing 62 may be shifted and/or modified to provide for improved viewing of the catheter tube 66.

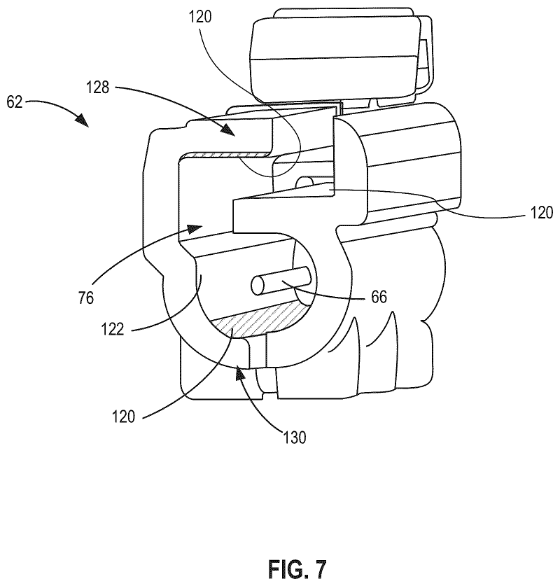

According to some embodiments, and as shown in FIGS. 6A and 6B and FIG. 7, at least one of housing portions 78a, 78b of housing 62 includes a paint, finish, or coating 120 (hereafter “coating 120”) applied thereto that enables improved viewing of the catheter tube 66. In such embodiments, each of housing portions 78a, 78b is formed of a transparent or translucent material 121 that provides for at least partial visibility into the inner volume 76 thereof, but at least one of housing portions 78a, 78b includes a coating 120 applied thereto—with the coating 120 preferably applied to the interior surface 122 of housing 62, but with it recognized that the coating could instead be applied to the exterior surface 124 of housing 62.

In one embodiment, the coating 120 applied to housing 62 (e.g., housing portion 78b) is an opaque coating that provides improved visibility of the catheter tube 66 and/or prevents light from entering the inner volume 76 where the catheter tube 66 is located.

In the embodiment of FIGS. 6A and 6B, the opaque coating 120 is applied to the interior surface 122 of one portion (e.g., housing portion 78a) of the housing, along a side region 126 thereof. In one embodiment, the opaque coating 120 may have a white or other light-hued coloration and, with the coating 120 applied to side region 126, the coating 120 serves as a background that provides improved contrast between the catheter tube 66 and the housing 62, so as to provide improved visibility of the catheter tube 66. In another embodiment, the coating 120 applied to the side region 126 of a housing portion (e.g., housing portion 78a) is a coating that acts as a one-way mirror that may be looked through from the near side, but has an opaque or reflective nature on the other side to enable improved visualization. When using the blood collection device 60, a clinician can better visualize the catheter tube 66 via the mirror-type coating 120 reflecting back images thereof.

In the embodiment of FIG. 7, the opaque coating 120 is applied to the interior surface 122 of the housing 62, along some surfaces of top and bottom regions 128, 130 of the housing portions 78a, 78b. With the coating 120 applied to some surfaces of the top and bottom regions 128, 130 of housing portions 78a, 78b the opaque coating 120 functions to prevent any light from entering the inner volume 76 where the catheter tube 66 is located. In some embodiments, the opaque coating 120 applied to the interior surface 122 of the housing 62 may have a matte finish that serves to further diffuse light within the inner volume 76 of the housing 62.

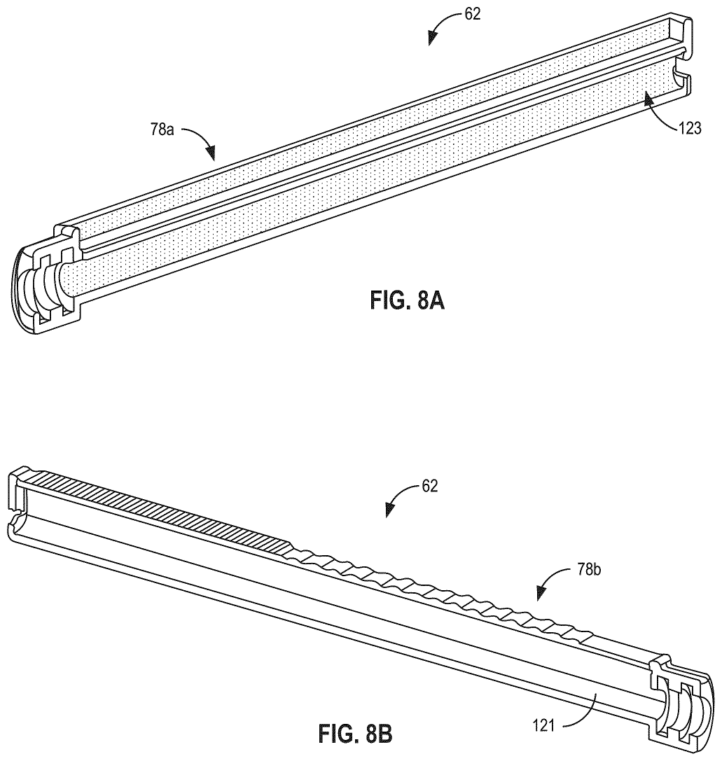

According to some embodiments, and as shown in FIGS. 8-17, the structure of the housing 62 and/or the material(s) from which housing 62 is constructed enable improved viewing of the catheter tube 66 within the interior of the housing 62.

As shown in FIGS. 8A and 8B, according to some embodiment, at least one of housing portions 78a, 78b is formed (i.e., molded) of a white or lighter colored material 123 that improves visibility of the catheter tube 66. In accordance with one aspect, one housing portion (e.g., housing portion 78a) is formed of a white or light-colored opaque material 123, while the other housing portion (e.g., housing portion 78b) is formed of a transparent or translucent material 121 that provides for visibility into the inner volume 76 of housing 62. The white or light-colored opaque material of housing portion 78a contrasts against a darker color of the catheter tube 66, so as to provide improved visibility of the catheter tube 66 when the housing 62 is rotated to a desired orientation where the opaque housing 62 serves as a background to the catheter tube 66.

According to another embodiment, each of housing portions 78a, 78b may be formed of a semi-transparent material having a white or light-colored tint thereto that contrasts against a darker color of the catheter tube 66, so as to provide improved visibility of the catheter tube 66. In such an embodiment, no particular rotation or orientation of the housing 62 is required, as the lightly tinted semi-transparent material of housing portions 78a, 78b may provide a background to the catheter tube 66 in multiple orientations.

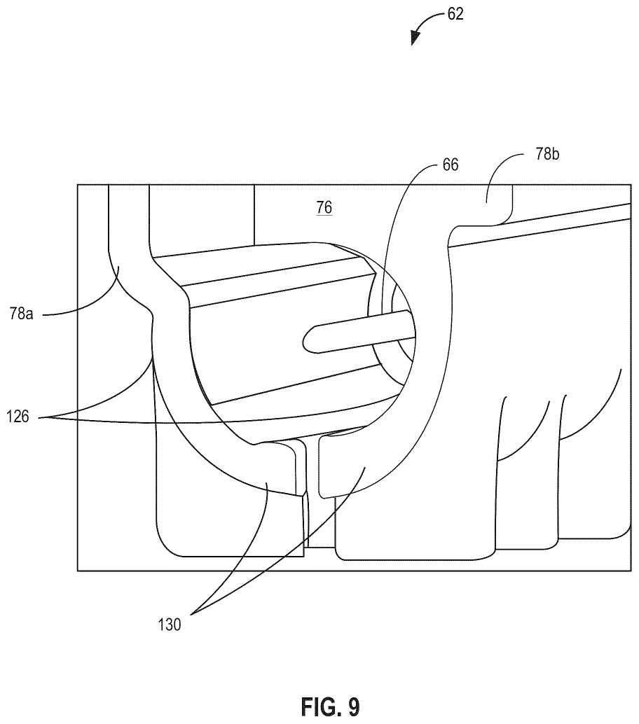

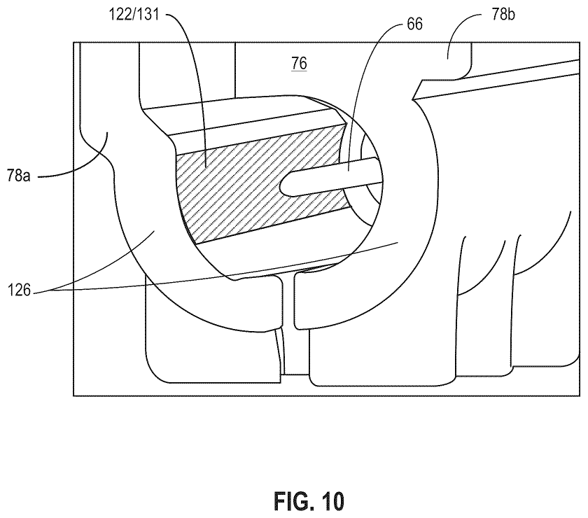

According to other embodiments, the housing 62 (i.e., housing portions 78a, 78b) is formed of a transparent or translucent material 121 that is modified to enable improved viewing of the catheter tube 66 within the inner volume 76 of the housing 62. That is, housing 62 may be structured such that at least the side region 126 of each of housing portions 78a, 78b is formed to increase visibility of the catheter tube 66 within housing 62. In one embodiment, and as shown in FIG. 9, the thickness of side regions 126 may be reduced (as compared to a top region 128 and bottom region 130 of housing 62) to facilitate better visualization. In another embodiment, and as shown in FIG. 10, a finish 131 of the interior surface 122 of the side region(s) 126 may be modified to reduce glare on the region(s).

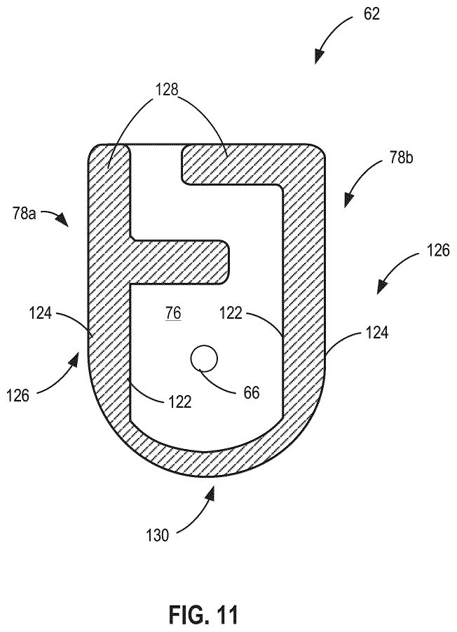

Referring now to FIGS. 11-15, in some embodiments, the shape of side regions 126 is controlled to improve visualization of the catheter tube 66 within the interior of the housing 62.

In one embodiment, and as shown in FIG. 11, the side regions 126 of housing 62 are structured as flat surfaces (instead of curved) to minimize glare and facilitate ease of visualization. That is, both an interior surface 122 and exterior surface 124 of the side regions 126 are formed as flat, so as to create a flat lens that may be oriented by a clinician during use of blood draw device 60, so as to remove a glare from the side regions 126. While a cylindrical shape of side regions 126 would act as a mirror that reflects any outside light source along its length (such that there would always be a portion of the housing 62 that will reflect the outside lights back to the eye of a user regardless of the housing orientation), forming the side regions 126 as flat sides will reflect light only in one direction, thereby allowing a user to rotate the housing 62 along its axis to direct the reflected light away from their eyes, to thereby improve visibility.

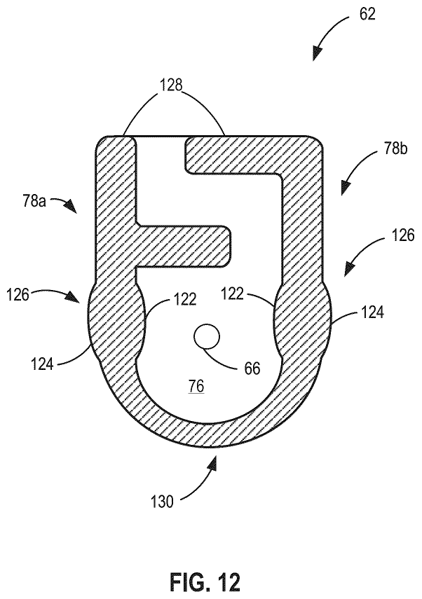

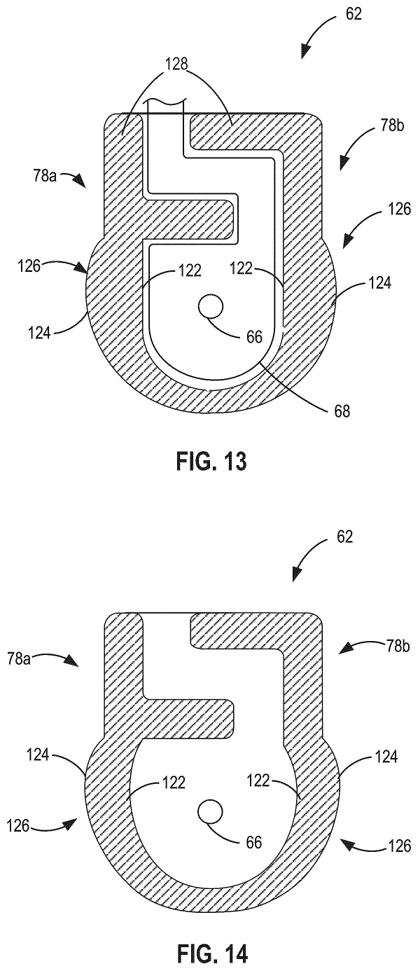

In other embodiments, and as shown in FIGS. 12 and 13, the interior surface 122 of one or both side regions 126 has a shape different from the exterior surface 124 thereof. In one configuration, and as shown in FIG. 12, the interior surface 122 of one or both side regions 126 has a convex shape, to add a magnifying effect to the visibility of the catheter tube 66 for easy visualization. In another configuration, and as shown in FIG. 13, the interior surface 122 of one or both side regions 126 has a flat shape (while exterior surface 124 remains curved), to again add a magnifying effect to the visibility of the catheter tube 66 for easy visualization. As further shown in FIG. 13, flat surfaces could also be provided on the sides of advancement member 68. In still another configuration, and as shown in FIG. 14, the exterior surface 124 of side regions may be curved at a smaller radius than the interior surface 122, with the smaller radius reducing glare from lights from broad lines to smaller lines and also adding a magnifying effect to the visibility of the catheter tube 66.

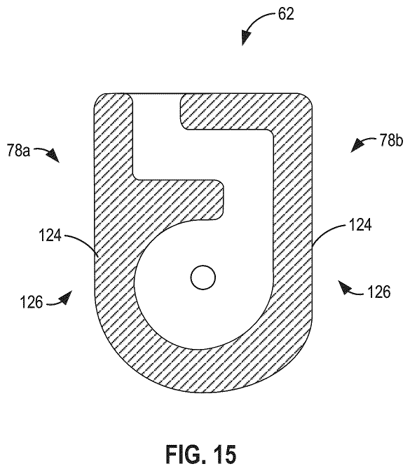

In another embodiment, and as shown in FIG. 15, interior and/or exterior surfaces 122, 124 of one or both side regions 126 are “smoothed out” to remove any steps, indents, or other extra surface features from the housing 62, so as to provide smooth surfaces on the housing 62. Removal of such extra surface features from the housing 62 minimizes the amount by which the housing 62 refracts and reflects light as it passes therethrough, therefore improving visibility of the catheter tube 66 within the housing 62.

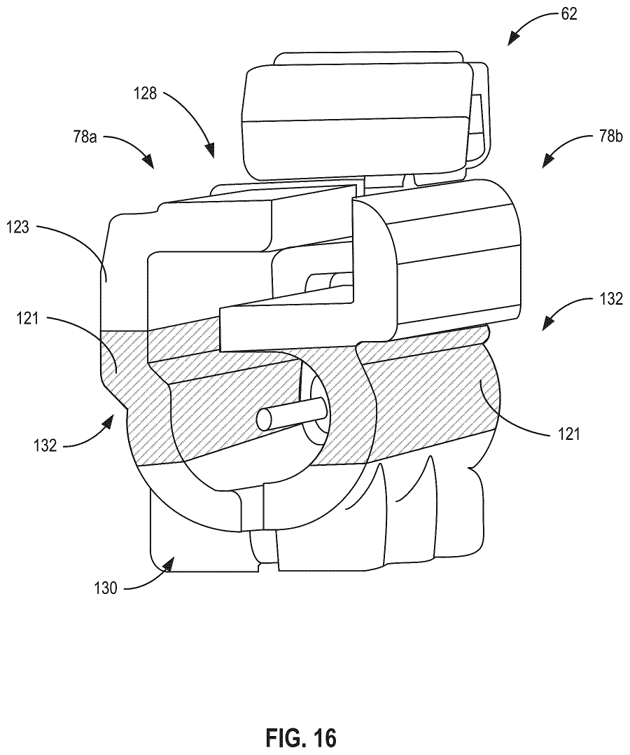

According to another embodiment, and as shown in FIG. 16, one or both of housing portions 78a, 78b is formed as a co-molded component that includes two different materials therein. That is, some sections/regions of one or both of housing portions 78a, 78b (e.g., top and bottom regions 128, 130 of each housing portion) may be formed of an opaque material 123, while other sections/regions of one or both of housing portions 78a, 78b (e.g., side regions or “windows” 132 of each housing portion) are formed of a clear or transparent material 121. In some embodiments, each of housing portions 78a, 78b is formed as a co-molded component with a transparent side window 132, such that two (2) opposing side windows 132 are formed in the housing 62 to provide visibility of the catheter tube 66. In other embodiments, only one of housing portions 78a, 78b is formed as a co-molded component with a transparent side window 132, such only a single side window 132 is formed in the housing 62 to provide visibility of the catheter tube 66.

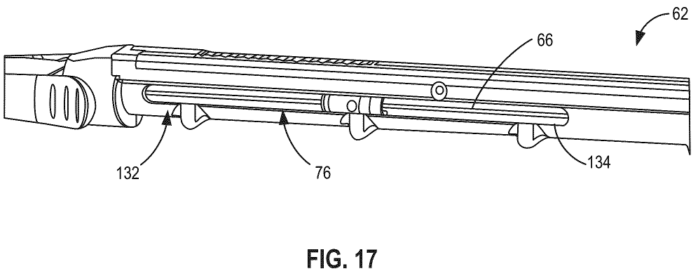

In some embodiments, and as shown in FIG. 17, rather than housing 62 being formed as a co-molded component with side windows 132 formed of a different material, housing 62 may instead be formed such that side windows are provided therein that are initially formed as open slots 134. The open side slots 134 may be formed along a full or partial length of the housing 62 and may have a transparent material 136 inserted therein, such as glass, a clear plastic insert, or a clear plastic film, that provides visibility into the inner volume 76 of housing 62 to enable visualization of the catheter tube 66.

According to other aspects of the disclosure, additional components may be added on or into the housing 62 that provide improved viewing of the catheter tube 66 during operation of the device 60.

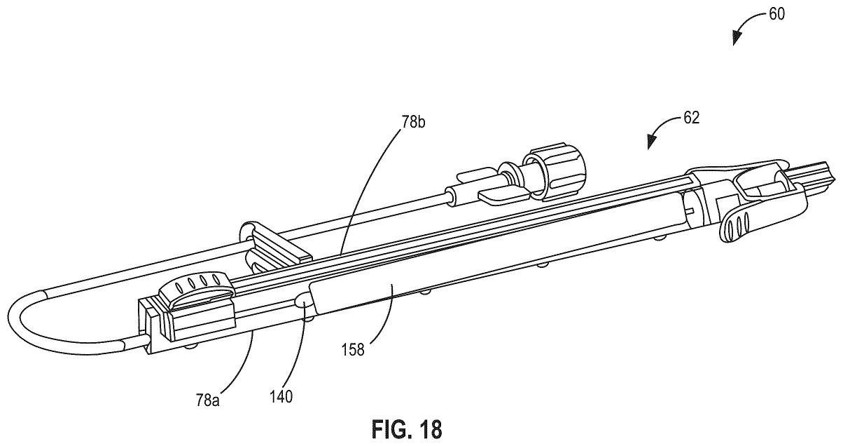

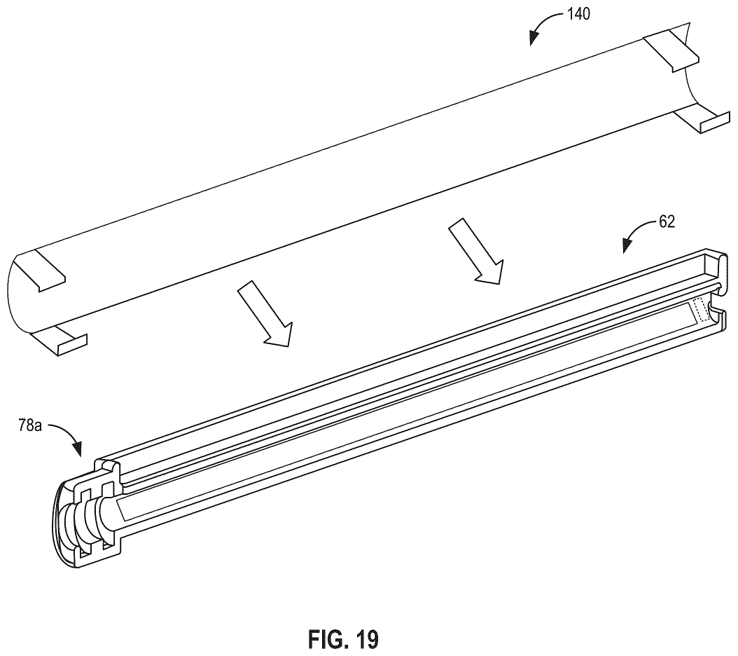

Referring to FIG. 18, according to one embodiment, a sticker 138 may be provided along with blood draw device 60 that may be applied to the housing 62 (by a clinician) at the time of use of the device 60 to enable improved visualization of the catheter tube 66. The sticker 138 may be configured as a white (or other light color) that may be applied onto a side region of the housing 62 (on either housing portion 78a, 78b) to provide a white background that is contrasted against the catheter tube 66 to improve visualization thereof. In one embodiment, the sticker 138 may initially be provided separate from the housing 62, such that the sticker 138 may be selectively applied to the desired housing portion 78a, 78b, thereby allowing the clinician to select which side to apply it to and allowing the clinician to only use the sticker 138 when needed. In another embodiment, stickers 138 may be pre-applied to both sides of the housing 62, with one of the stickers 138 then being removed by the clinician from a desired side of the housing 62, to provide for visualization of the catheter tube 66 through a desired side of the housing 62. In some embodiments, a tab 140 could be provided on the stickers 138 to enable easier removal thereof from the housing 62.

Referring to FIG. 19, according to another embodiment, a sleeve 140 may be provided separate from housing 62 that can be snapped onto the housing 62 (by a clinician) at the time of use of the device 60 to enable improved visualization of the catheter tube 66. The sleeve 140 may be snapped onto a side region of either housing portion 78a, 78b (portion 78a in FIG. 19), thereby enabling right- or left-handed use of the blood draw device 60. The sleeve 140 may be configured as a white (or other light color) sleeve 140 that provides a light-colored background that is contrasted against a darker catheter tube 66 to improve visualization thereof. Alternatively, the sleeve 140 may be configured as a darker colored sleeve 140 that provides a dark background that is contrasted against a white or other light-colored catheter tube 66 to improve visualization thereof.

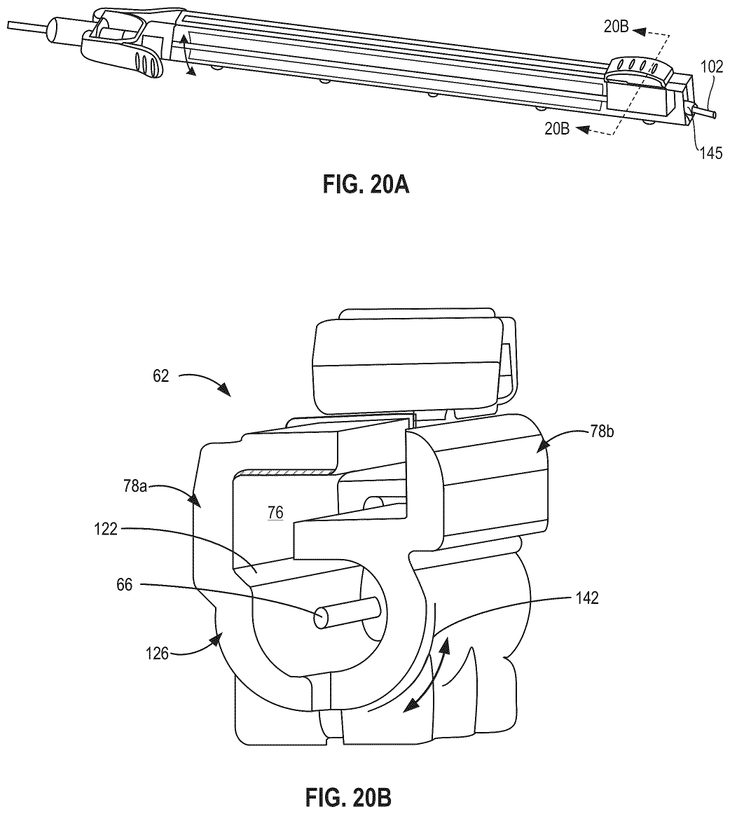

Referring to FIGS. 20A and 20B, according to another embodiment, an insert 142 may be provided separate from housing 62 that is permanently affixed on an outer surface thereof or within the inner volume 76 thereof and that is movable between different positions (by a clinician) at the time of use of the device 60 to enable improved visualization of the catheter tube 66. The insert 142 may generally conform to a portion of an exterior (or interior) surface 122 of housing 62, such as to a side region 126 of the housing. The insert 142 may be configured as a white (or other light color) insert 142 that provides a light background that is contrasted against the darker catheter tube 66 to improve visualization thereof. In use of the blood draw device 60, a clinician may position the insert 142 at a desired location (e.g., at a right side position or a left side position)—with movement of the insert 142 being provided via a twist knob 145 located on an external surface of the housing 62, such as on a proximal end surface of the housing 62 and encircling the secondary catheter 102. The clinician may turn the twist knob 145 to translate the insert 142 to a desired side of housing 62, with the insert 142 functioning to shield light out from housing 62 and provide a contrasting background for visualization of the catheter tube 66.

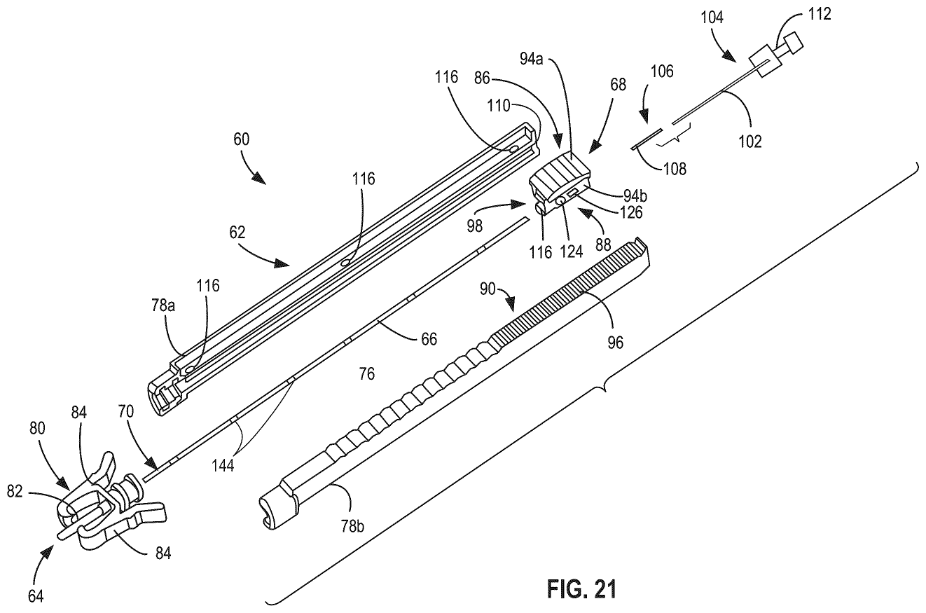

According to other aspects of the disclosure, the catheter tube 66 may be configured to provide improved viewing thereof within housing 62 during operation of the device 60. According to some embodiments, the catheter tube 66 may be provided such that at least portions thereof are colored black or another dark color that contrasts against a lighter (e.g., white) colored background provided in the housing 62 (according to any of previous embodiments described herein). An entirety of the catheter tube 66 may be colored black/dark in some embodiments, such as by forming the tube out of a black/dark polyimide material, while in other embodiments black stripes 144 may be provided on the catheter tube 66, such as by painting black stripes 144 on the polyimide tube 66 at various locations, with each of the stripes 144 extending circumferentially about the catheter tube 66, as shown in FIG. 21.

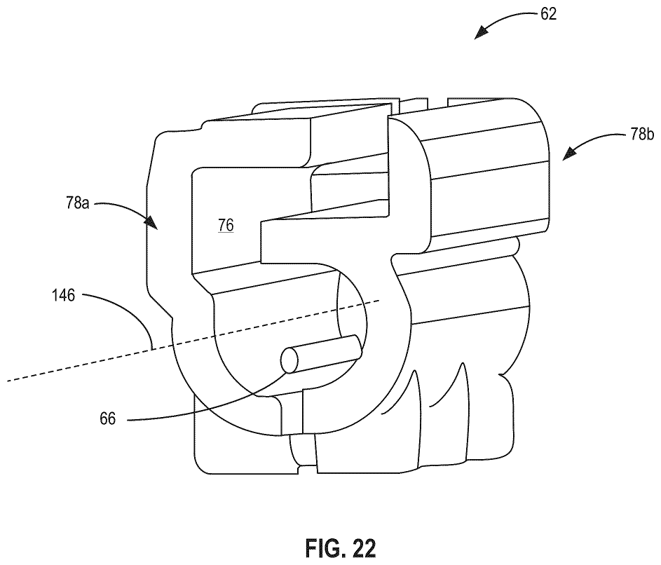

According to some embodiments, in order to provide improved viewing of the catheter tube 66 within housing 62, and as shown in FIG. 22, the catheter tube 66 may be positioned off-center within the inner volume 76 of the housing 62 (relative to a center line 146 of the housing 62 extending longitudinally therealong). That is, it is recognized that when the catheter tube 66 is centered within the inner volume 76, the catheter tube 66 may be positioned in a region of the inner volume 76 where optical glare from the housing 62 negatively impacts visualization of the catheter tube 66. Accordingly, the catheter tube 66 may be positioned off-center within the inner volume 76 of the housing 62 relative to center line 146 in a region where the optical glare is reduced, therefore making it easier to visualize the catheter tube 66 within housing 62.

Therefore, according to embodiments described herein, the blood draw device 60 is configured to provide for improved viewing of the catheter tube 66 as the catheter tube 66 is advanced into and through a catheter assembly and into the vein of a patient. Visualization aiding elements are provided in blood draw device 60 that are configured to improve visibility into the inner volume 76 of housing and of the catheter tube 66 therein during operation of the blood draw device 60, such that a condition (e.g., bowing or kinking) and positioning of the catheter tube 66 can be determined as the catheter tube 66 is advanced and/or retracted relative to the housing 62 and into catheter assembly 12.

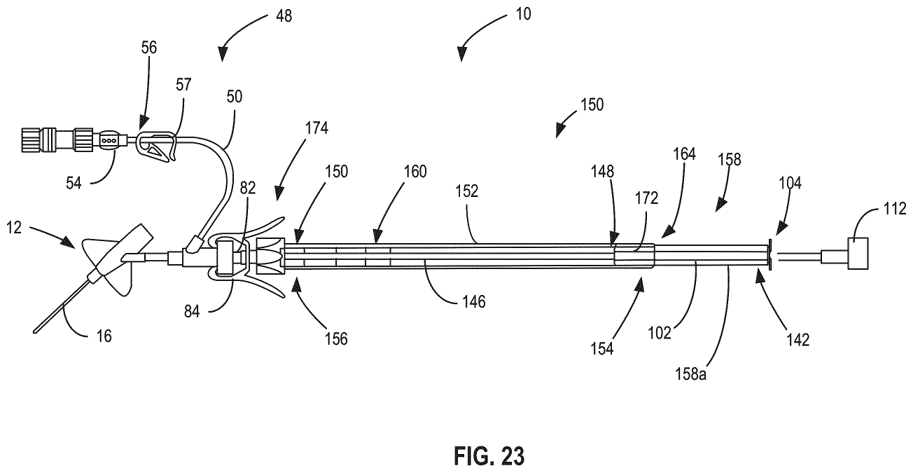

It is recognized that aspects of the disclosure are not limited to the specific blood draw device 60 shown and described in FIGS. 1-22, and that blood draw devices with other suitable constructions may also incorporate aspects of the disclosure. Referring now to FIG. 23, a catheter system 10 is shown that includes a blood draw device 150 according to another embodiment of the disclosure. The blood draw device 150 includes a housing 152 having a proximal end 154 and a distal end 156, and an advancement member 158 slideably received within housing 152 (i.e., within an inner volume 160 of housing 152). In the illustrated embodiment, the advancement member 158 is provided as one or more telescopic cylinders 158a that are provided in a telescoping relationship with housing 152, such that advancement member 158 may be slideably received entirely, or almost entirely, within the inner volume 160 of housing 152. Advancement member 158 also includes a proximal end 162 and a distal end 164 and, in non-limiting embodiments, advancement member 158 may have a variable diameter along its length. As one example, the distal end 164 of advancement member 158 may have a larger diameter than other portions of advancement member 158 such that, as advancement member 158 is retracted, one or more features on housing 152 may interact with the enlarged portion of advancement member 158 to prevent pulling advancement member 158 completely out of housing 152. As another example, the distal end 164 of advancement member 158 may have a smaller diameter than other portions of advancement member 158, to keep the advancement member 158 in position at a blood draw forward condition, so that a hand of the operator is freed up to manipulate additional components (e.g., a vacutainer tube).

Blood draw device 150 further includes a catheter tube 166 having a proximal end 168 and a distal end 170. Catheter tube 166 is received within the inner volume 160 of housing 152, and may be advanced and/or retracted relative to housing 152 by displacement of the advancement member 158 relative to the housing 152. In some embodiments, the catheter tube 166 may be joined to advancement member 158 via a fitting 172 provided at the distal end 164 of advancement member 158, such that displacement of the advancement member 158 relative to the housing 152 causes a corresponding displacement of catheter tube 166. In non-limiting embodiments, catheter tube 166 may be advanced from a first positon in which distal end 170 of catheter tube 166 is within housing 152, to a second position in which a distal end 170 of catheter tube 166 is positioned distally of housing 152 (and also positioned distally of catheter 16), as previously described regarding blood draw device 60 and operation thereof.

Blood draw device also includes a coupling device 174 thereon which may be identical to the coupling device 64 shown and described in the blood draw device of FIGS. 1-12. That is, coupling device 174 is configured as a lock 80 that includes a blunted cannula 82 and locking arms 84 for coupling to the needleless access connector 46 of catheter assembly 12, with the blunted cannula 82 and locking arms 84 forming three points of contact therewith. However, it is appreciated that alternative embodiments of blood draw device 150 may include a coupling device 174 of another type to secure blood draw device 150 to catheter assembly 12, including luer connections, clips, blunt plastic cannulae, blunt metal cannulae, hybrid luers (e.g., with a cannula) friction fits, and the like. According to aspects of the disclosure, a secondary catheter 102 may be routed through the telescopic cylinder 158a of advancement member 158, with the secondary catheter 102 providing a fluid connection between the catheter tube 166 and a coupler 112 provided at the proximal end portion 104 of the secondary catheter 102—with the coupler 112 configured to mate with a collection device (not shown).

As described in detail above, blood draw device 150 may incorporate one or more visualization aiding elements therein that are configured to improve visibility of the catheter tube 166 during operation of the blood draw device 150, such that a condition (e.g., bowing/kinking) and positioning of the catheter tube 166 can be determined as the catheter tube 166 is advanced and/or retracted relative to the housing 152 and into catheter assembly 12. In accordance with aspects of the disclosure, visualization of the catheter tube 166 may be improved by modifying the housing 152 (e.g., modifying the structure of the housing 152, the material from which housing 152 is formed, and/or applying coating(s) to the housing 152), adding components onto the housing 152, and/or by shifting/modifying the catheter tube 166 within the housing 152. According to embodiments, any of the visualization aiding elements shown in the embodiments of FIGS. 6-22 may be incorporated into the blood draw device 150 of FIG. 23.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. An instrument delivery device for use with an intravenous catheter assembly, the instrument delivery device comprising:
a housing having a proximal end portion and a distal end portion, the housing comprising a first housing portion and a second housing portion, with the housing portion and the second housing portion arranged side-by-side and coupled together to define an inner volume;
an instrument movably received within the inner volume;
a coupling device positioned at the distal end portion of the housing and configured to couple the housing to an access connector of an intravenous catheter assembly; and
an advancement member configured to move relative to the housing to advance a distal end of the instrument beyond the distal end portion of the housing and into the intravenous catheter assembly;
wherein the housing comprises a visualization aid configured to improve visibility of the instrument within the inner volume of the housing.

2. The instrument delivery device of claim 1, wherein each of the first housing portion and the second housing portion is formed of a transparent or semi-transparent material, with each of the first housing portion and the second housing portion comprises a top region, a bottom region, and a side region.

3. The instrument delivery device of claim 2, wherein the visualization aid comprises a coating applied to the first housing portion, the coating applied to an interior surface of the first housing portion on at least the side region.

4. The instrument delivery device of claim 3, wherein the coating comprises an opaque coating having a first color, and wherein the instrument has a second color that is darker than the first color, such that the coating is contrasted from the instrument.

5. The instrument delivery device of claim 3, wherein the coating forms a one-way mirror that may be looked through from a first side, but has an opaque or reflective nature on an opposing second side.

6. The instrument delivery device of claim 2, wherein the visualization aid comprises the side region of each of the first housing portion and the second housing portion.

7. The instrument delivery device of claim 6, wherein the side region of each of the first and second housing portions has a thickness that is less than a thickness of the top region and the bottom region, to facilitate better visualization of the instrument through the side region.

8. The instrument delivery device of claim 6, wherein the side region of each of the first and second housing portions has an interior surface and an exterior surface, with each of the interior surface and an exterior surface comprising a flat surface.

9. The instrument delivery device of claim 6, wherein the side region of each of the first and second housing portions has an interior surface and an exterior surface, with the exterior surface comprising a curved surface and the interior surface comprising a flat surface or a convex surface having a larger radius than the curved exterior surface.

10. The instrument delivery device of claim 6, wherein the side region of each of the first and second housing portions comprises a smoothed exterior surface free of any steps or indents, the smooth exterior surface minimizing refraction and reflecting of light as it passes through the side region.

11. The instrument delivery device of claim 2, wherein the visualization aid comprises a sticker applied to the side region of the first housing portion, the sticker applied to an exterior surface of the side region and having a first color, and wherein the instrument has a second color that is darker than the first color, such that the instrument is contrasted from the sticker.

12. The instrument delivery device of claim 2, wherein the visualization aid comprises a sleeve secured to an exterior surface of the first housing portion, the sleeve having a first color, and wherein the instrument has a second color that is darker than the first color, such that the instrument is contrasted from the sleeve.

13. The instrument delivery device of claim 2, wherein the visualization aid comprises an insert positioned adjacent an interior surface of the housing, within the inner volume, the insert having a first color, and wherein the instrument has a second color that is darker than the first color, such that the instrument is contrasted from the insert, and wherein the insert is movable from a first position adjacent the side region of the first housing portion to a second position adjacent the side region of the second housing portion via a twist knob located on the housing.

14. The instrument delivery device of claim 1, wherein the visualization aid comprises an opaque material from which the first housing portion is formed, with the second housing portion formed of a transparent or semi-transparent material, the opaque material having a first color, and wherein the instrument has a second color that is darker than the first color, such that the first housing portion is contrasted from the instrument.

15. The instrument delivery device of claim 1, wherein the visualization aid comprises a semi-transparent material from which the first housing portion and the second housing portion are formed, the semi-transparent material having a tinted first color, and wherein the instrument has a second color that is darker than the first color, such that the first housing portion and the second housing are contrasted from the instrument.

16. The instrument delivery device of claim 1, wherein at least one of the first housing portion and the second housing portion comprises a co-molded component having a top region, a bottom region, and a side region, wherein the top region and the bottom region are formed of an opaque material and the side region is formed of a transparent material; and
wherein the visualization aid comprises the side region.

17. The instrument delivery device of claim 1, wherein each of the first housing portion and the second housing portion is formed of an opaque material, with each of the first housing portion and the second housing portion comprising a top region, a bottom region, and a side region; and
wherein the visualization aid comprises a window formed in the side region of at least one of the first housing portion and the second housing portion, with a transparent member secured within the window.

18. An instrument delivery device for use with an intravenous catheter assembly, the instrument delivery device comprising:
a housing having a proximal end portion and a distal end portion, the housing comprising a first housing portion and a second housing portion, with the housing portion and the second housing portion arranged side-by-side and coupled together to defining an inner volume;
an instrument movably received within the inner volume;
a coupling device positioned at the distal end portion of the housing and configured to couple the housing to an access connector of an intravenous catheter assembly; and
an advancement member configured to move relative to the housing to advance a distal end of the instrument beyond the distal end portion of the housing and into the intravenous catheter assembly; and
wherein the instrument comprises a visualization aid configured to improve visibility of the instrument within the inner volume of the housing.

19. The instrument delivery device of claim 18, wherein the visualization aid comprises a plurality of stripes formed on the instrument, each of the plurality of stripes extending circumferentially about the instrument.

20. The instrument delivery device of claim 18, wherein the visualization aid comprises a coloring of the instrument, wherein the coloring of instrument is darker than a coloring of the housing, such that the instrument is contrasted from the housing.

21. The instrument delivery device of claim 18, wherein the visualization aid comprises a positioning of the instrument within the inner volume of the housing, the instrument positioned so as to be off-center relative to a center line of the inner volume extending longitudinally between the distal end and the proximal end of the housing.
